(19) United States
(12) Patent Application Publication
(10) Pub. No.: US 2025/0099718 A1
(43) Pub. Date: Mar. 27, 2025

(54) Catheter Placement Device Including an Extensible Needle Safety Component

(71) Applicant: C. R. Bard, Inc., Franklin Lakes, NJ (US)

(72) Inventor: Huy Ngoc Tran, Riverton, UT (US)

(21) Appl. No.: 18/972,436

(22) Filed: Dec. 6, 2024

Related U.S. Application Data
(62) Division of application No. 17/353,602, filed on Jun. 21, 2021, now Pat. No. 12,161,819.
Application No. 17/353,602 is a division of application No. 15/154,384, filed on May 13, 2016, now Pat. No. 11,040,176.
(60) Provisional application No. 62/162,548, filed on May 15, 2015.

(51) Int. Cl. A61M 25/06

(52) U.S. Cl. CPC A61M 25/0606; A61M 25/0618; A61M 25/0693; A61M 2205/586

(57) ABSTRACT

An insertion device for inserting a catheter into a patient's body can include needle insertion, guidewire advancement, catheter insertion, and needle shielding in a single device. The insertion device can include a housing with a needle distally extending therefrom. A catheter can be pre-disposed over the needle such that the catheter is disposed substantially external to the housing. A guidewire can be included, as well as an advancement assembly that is configured to selectively advance the guidewire out of a distal opening of the needle. The needle of the insertion device can include a flash indicator configured to indicate the presence of fluid within the needle lumen. The flash indicator can include an absorbent material disposed about the needle. The absorbent material can include a tether. The insertion device can include a needle safety component. The safety component can include a locking element and a cam component.

### CROSS-REFERENCE TO RELATED APPLICATIONS

This application is a division of U.S. patent application Ser. No. 17/353,602, filed Jun. 21, 2021, now U.S. Pat. No. 12,161,819, which is a division of U.S. patent application Ser. No. 15/154,384, filed May 13, 2016, now U.S. Pat. No. 11,040,176, which claims the benefit of priority to U.S. Provisional Application No. 62/162,548, filed May 15, 2015, each of which is incorporated by reference in its entirety into this application.

### BRIEF SUMMARY

Briefly summarized, embodiments of the present invention are directed to an insertion device for inserting a catheter or other tubular medical device into a body of a patient. The insertion device combines needle insertion, guidewire advancement, catheter insertion, and needle shielding in a single device. In one embodiment, the insertion device comprises a housing and a hollow needle that distally extends from the housing. At least a portion of the catheter is pre-disposed over the needle such that the catheter is disposed substantially external to the housing. A guidewire is included, as well as an advancement assembly that is configured to selectively advance the distal end of the guidewire out a distal opening of the needle in preparation for distal advancement of the catheter. The advancement assembly is further configured to enable selective advancement of the catheter in a distal direction. The insertion device is configured to be grasped and used by a single hand of a user during advancement of the guidewire and the catheter.

In another embodiment, continuous blood flash indicators are disclosed to assist in confirming that the needle of the catheter insertion device has accessed and remains in a vein or other blood-carrying vessel. In yet another embodiment, needle safety components are disclosed for use with the catheter insertion device.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A-1J show various views of a catheter insertion tool according to one embodiment;

FIGS. 2A-2C show various views of components of the catheter insertion tool of FIGS. 1A-1J;

FIGS. 3A-3C show various stages of use of the catheter insertion tool of FIGS. 1A-1J;

FIG. 4 is a cross-sectional side view of a blood flash indicator according to one embodiment;

FIG. 5 is a perspective view of a blood flash indicator according to one embodiment;

FIG. 6 is a top view of a blood flash indicator according to one embodiment;

FIG. 7 is a top view of a blood flash indicator according to one embodiment;

FIGS. 8A-8H are various views of a catheter insertion tool according to one embodiment;

FIGS. 9A-9G are various views of a needle safety component according to one embodiment;

FIG. 10 is a cross-sectional view of a needle and guidewire according to one embodiment;

FIG. 11 is a cross-sectional view of a needle and guidewire according to one embodiment;

FIG. 12 is a cross-sectional view of a needle and guidewire according to one embodiment;

FIG. 13 is a partial cross-sectional side view of a needle and guidewire according to one embodiment;

FIG. 14 is a partial cross-sectional side view of a needle and guidewire according to one embodiment;

FIG. 15 is a partial cross-sectional side view of a needle and guidewire according to one embodiment;

FIG. 16 is a partial cross-sectional side view of a needle and guidewire according to one embodiment;

FIG. 17 is a side view of a guidewire according to one embodiment;

FIG. 18 is a perspective view of an advancement member of a catheter insertion tool according to one embodiment;

FIG. 19 is a top view of a blood flash indicator according to one embodiment;

FIG. 20 is a top view of a blood flash indicator according to one embodiment;

FIG. 21 is a top view of a blood flash indicator according to one embodiment;

FIG. 22 is a top view of a blood flash indicator according to one embodiment;

FIG. 23 is a top view of a blood flash indicator according to one embodiment;

FIG. 24 is a top view of a blood flash indicator according to one embodiment;

FIG. 25 is a top view of a blood flash indicator according to one embodiment;

FIGS. 26A-26D are various views of a catheter insertion tool according to one embodiment;

FIGS. 27A-27E are various views of a catheter insertion tool according to one embodiment;

FIG. 28 is a perspective view of a blood flash indicator according to one embodiment;

FIG. 29 is a perspective view of a catheter insertion tool according to one embodiment; and

FIGS. 30A and 30B depict various views of a blood flash indicator according to one embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

Embodiments of the present invention are generally directed to a tool for assisting with the placement into a patient of a catheter or other tubular medical device. For example, catheters of various lengths are typically placed into a body of a patient so as to establish access to the patient's vasculature and enable the infusion of medicaments or aspiration of body fluids. The catheter insertion tool to be described herein facilitates such catheter placement. Note that, while the discussion below focuses on the placement of catheters of a particular type and relatively short length, catheters of a variety of types, sizes, and lengths can be inserted via the present device, including peripheral IVs, intermediate or extended-dwell catheters, PICCs, central venous catheters, etc. In one embodiment, catheters having a length between about 1 inch and about 1.9 inches can be placed, though many other lengths are also possible.

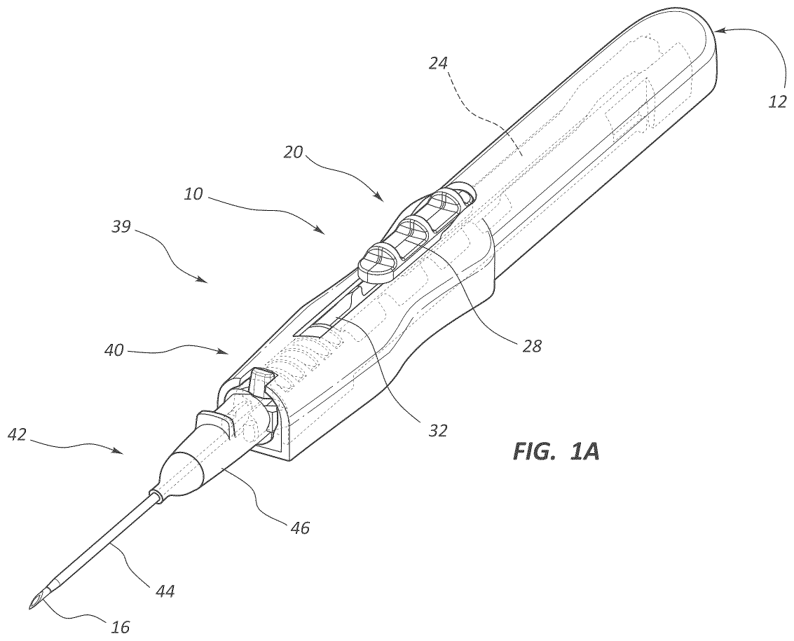

FIGS. 1A-1J depict various details regarding a catheter insertion tool (“insertion tool” or “insertion device”), generally depicted at 10, according to one embodiment. As shown, the insertion tool 10 includes a housing 12 that may itself include a top front housing portion 12A, a top back housing portion 12B, and a bottom housing portion 12C mated with one another via tabs and slots 78 or other suitable attachment modes. The housing 12 further includes an open distal end 12D, and a flat bottom 12E to enable the insertion device 10 to lie flat on a surface without tipping. In another embodiment, the housing is integrally formed. In yet another embodiment, only a top housing portion and a bottom housing portion are employed. In the present embodiment, the housing composed of a thermoplastic such as polycarbonate and is translucent, though other configurations are contemplated. A rib 38 runs along the longitudinal length of the bottom housing portion 12C, in the present embodiment. The housing 12 defines grip surfaces 74 on either side of the housing, as seen in FIGS. 1B and 1G, to enable grasping of the insertion device 10 by the user.

A needle hub 14 supporting a hollow needle 16 (which together form part of a needle assembly, in one embodiment) is included with the housing 12. In the present embodiment, the needle hub 14 is integrally formed with the housing 12 within a cavity 70 defined by the housing, as best seen in FIG. 1I, though the needle hub can be configured in other ways. The needle hub 14 includes a pocket 14A for receiving a portion of the needle 16 and a quantity of adhesive, such as liquid or UV-cure adhesive for example, in order to fix the needle in place in the needle hub. The needle 16 extends distally from the needle hub 14 so as to extend out an open distal end 12D of the housing 12 and terminates at a distal end 16B. A notch 18 is defined through the wall of the needle 16 proximate the distal end thereof. The notch 18 enables flashback of blood to exit the lumen defined by the hollow needle 16 once access to the patient's vasculature is achieved during catheter insertion procedures. Thus, blood exiting the notch 18 can be viewed by a clinician to confirm proper needle placement in the vasculature, as will be explained further below.

A catheter 42 is removably disposed on the portion of the needle 16 residing external to the housing 12 such that the needle occupies a lumen of the catheter defined by a catheter tube 44. The catheter tube 44 extends distally from a hub 46 of the catheter 42, which hub is initially disposed adjacent the open distal end 12D of the housing 12, as shown in FIGS. 1A-1C.

The insertion tool 10 further includes a guidewire advancement assembly 20 for advancing a guidewire 22 through the needle 16 and into the vasculature of the patient once access by the needle has been achieved. The guidewire 22 (FIG. 1I) is pre-disposed within the lumen of the needle 16. The guidewire advancement assembly 20 includes a guidewire lever 24 that selectively advances the guidewire 22 in a distal direction during use of the insertion tool 10 such that the distal portion of the guidewire extends beyond the distal end 16B of the needle 16. A finger pad 28 of the guidewire lever 24 is slidably disposed on the housing 12 via a slot 32 to enable a thumb and/or finger(s) of the user to selectively advance the guidewire 22 distally past the distal end 16B of the needle 16. Of course, other engagement schemes to translate user input to guidewire movement could also be employed. In the present embodiment, the guidewire 22 includes a guidewire support tube 19 (FIGS. 1I, 1J) to provide additional stiffness to the guidewire and facilitate its distal advancement described above.

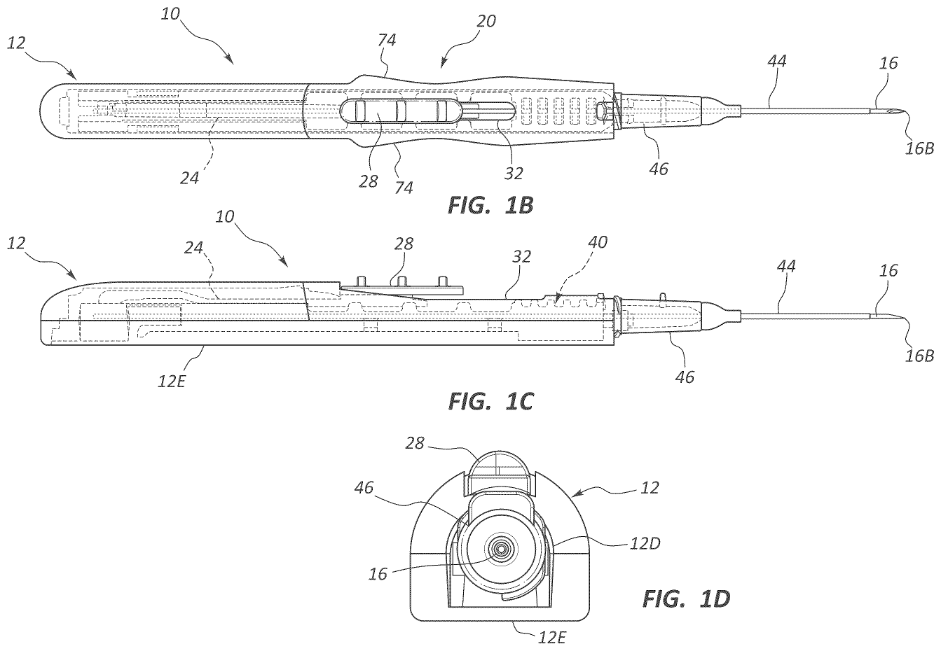

Together with FIG. 1H, reference is made to FIGS. 2A-2C in describing further details of the insertion tool 10 of FIGS. 1A-1J. Further details of the guidewire lever 24 are shown, including a lever tab 26 at the proximal end of the guidewire lever that is used as described below in distally advancing the guidewire 22. An angled portion 24A of the guidewire lever 24 is disposed distal to the lever tab 26. A push tab 30 is defined below the finger pad 28 and is employed during distal advancement of the guidewire lever 24 to partially advance the catheter 42 distally, as will be described below.

In the present embodiment, a proximal end of the guidewire 22 is attached at an anchor point 36 on an interior portion of the housing 12 (or other fixed portion of the insertion tool 10) and looped about the proximal portion of the guidewire lever 24 in a roughly U-shaped configuration (FIG. 1H) such that the distal end of the guidewire extends two units of distance distally past the distal end 16B of the needle 16 for every one unit of distance of movement of the finger pad 28.

In greater detail, FIGS. 2A-2C show that curved guide surfaces 34 are defined on the proximal end of the guidewire lever 24 to enable an intermediate portion of the guidewire 22 to loop back on itself proximate the proximal end of the device 10. The guide surfaces 34 constrain the flexible guidewire 22 into the looped, substantially U-shaped configuration. The angled portion 24A of the guidewire lever 24 helps to maintain the radius of the U-shaped portion of the guidewire 22 large enough so as to prevent kinking/undesired bending of the guidewire as it loops back. The looped-back intermediate portion of the guidewire 22 then extends toward the distal end of the device 10 within the cavity 70 of the housing 12 before it enters the lumen of the needle 16 at a proximal end of the needle, which is secured by the needle hub 14. A bar 34A is positioned across the guide surfaces 34 in the present embodiment to keep the guidewire 22 in contact with the guide surfaces. In one embodiment, the rib 38 can include a channel in which a portion of the intermediate portion of the looped guidewire 22 can reside so as to guide the guidewire toward the proximal end of the needle 16.

So configured, the free distal end of the guidewire 22 initially resides within the lumen of the needle 16 and is positioned for selective advancement by the guidewire advancement assembly 20 such that the free distal end thereof can distally extend from the open distal tip of the needle 16. This selective advancement of the guidewire 22 is achieved in the present embodiment via distal movement of the finger pad 28 included on the device housing 12. Distal movement of the finger pad 28 causes corresponding distal sliding movement of the guidewire lever 24. The guide surfaces 34 of the guidewire lever 26 push the bend of the guidewire 22 distally as the guidewire lever 24 advances. Note that the guidewire 22 is sufficiently rigid, in part due to the guidewire support tube 19, so as to be advanced by the guidewire lever 24 without buckling. Also, the guide surfaces 34 and guidewire 22 are configured to enable retraction of the guidewire 22 back into the insertion device housing 12 when the finger pad 28 (or other suitable mechanism in other embodiments) is slid proximally.

This distal sliding movement of the guidewire lever 24 causes the distal end of the guidewire 22 to extend distally from the open distal tip 16B of the needle 16. Because of its anchored proximal end at the anchor point 36 and its bent or looped U-shape configuration (FIG. 1H), the guidewire 22 is distally advanced at a linear rate of about twice the linear advancement rate of finger pad 28, which results in about twice the length of guidewire extension when compared with the length of movement of the guidewire advancement slide 28. This further desirably results in a relatively longer length of guidewire extension into the vein or other patient vessel so as to more suitably guide the catheter 42 into the patient's body. As such, the guidewire advancement assembly described here operates as a type of “reverse pulley” system for distal guidewire advancement. Note that other looping configurations of the guidewire can be included with the device 10 in addition to those shown and described herein. Also, differing ratios of guidewire extension vs. advancement assembly movement are also possible in other embodiments.

Note that the above-described structures for providing a looping guidewire are only examples of structures that can suitably perform the desired functionality described herein. Indeed, other structures can be employed to accomplish the principles described in connection with the present embodiment. Also, though shown and described above to be attached to the catheter insertion device housing, the proximal end of the guidewire can be attached to other structures within/on the device, such as the needle hub 14, for instance. The majority length of the guidewire in one embodiment includes a metal alloy of nickel and titanium commonly referred to as nitinol, which is sufficiently rigid and can be disposed in the U-shaped configuration without retaining a memory of that position when the guidewire is advanced. Note that other suitable guidewire materials can also be employed.

The insertion tool 10 further includes a catheter advancement assembly 40 for selectively advancing in a distal direction the catheter 42, pre-disposed on the needle 16 external to the housing 12. In particular, the catheter advancement assembly 40 includes an advancement member 48 that initially resides within the cavity 70 defined by the housing 12 and is used in selectively advancing the catheter 42 in a distal direction during use of the insertion tool 10 to insert the catheter into the body of the patient. As will be seen, the advancement member 48 also acts as a needle safety component for shielding the needle 16 from the user after use of the device 10 is complete, as will be described further below.

FIGS. 2A-2C depict further details of the advancement member 48 of the catheter advancement assembly 40 according to the present embodiment. As shown, the advancement member 48 defines an elongate body that is configured to straddle the needle 16 when the advancement member 48 is initially disposed within the housing cavity 70. The body of the advancement member 48 includes a distal portion 49. A pair of hub engagement tabs 50 extend distally from the distal end of the distal portion 49, and each engagement tab includes a radially extending protuberance 52. When the advancement member 48 is disposed within the housing 12, the needle 16 passes distally between the engagement tabs 50, as seen in FIG. 1H. The advancement member 48 is configured such that the needle 16 extends the engagement tabs 50 a relatively small distance radially outward while the needle is disposed therebetween. The catheter 42, when disposed over the needle 16, as seen in FIG. 1H, is kept in place against the open housing distal end 12D via the protuberances 52 of the engagement tabs 50 producing a friction fit against an inner surface of the catheter hub 46. As will be seen further below, when the needle 16 is no longer disposed between the engagement tabs 50, the tabs withdraw radially inward, allowing the catheter to separate from the distal end of the advancement member 48.

The distal portion 49 of the advancement member 48 further includes an advancement tab 54 for assisting with manual distal extension of the advancement tab during deployment of the catheter 42, and a plurality of top and bottom ribs 56 that extend across the advancement member body to join two elongate arms 58 that longitudinally extend proximally from the distal portion. FIGS. 1G and 1H show that the arms 58 straddle the needle 16 when the advancement member 48 is initially disposed in the cavity 70 of the housing 12. The top and bottom ribs 56 are positioned such that the distal tip 16B of the needle 16 is shielded from user contact after extension of the advancement member 48 from the housing 12 is complete, as discussed further below. In addition, a proximal end 56A of the ribs 56 (FIG. 1H) acts as a stop against a pair of lock wedges 72, formed in the housing 12, to prevent further proximal entry of the advancement member 48 into the housing cavity 70.

Each of the arms 58 includes secondary tabs 60 disposed proximal to the distal portion 49 to assist, together with the advancement tab 54, in manually extending the advancement member 48 in the distal direction by enabling locations for a finger of the user to push against. Proximate the proximal end of each advancement member arm 58, a locking tab 62 is included. The locking tabs 62 are deformable to enable them to pass over the lock wedges 72 when the advancement member 48 is distally extended during catheter distal advancement (described below) so as to prevent re-entry of the advancement member into the housing cavity 70, which also ensures that the distal tip 16B of the needle 16 remains shielded by the ribs 56 of the advancement member distal portion 49. In addition, stop surfaces 64 are included on the advancement member arms 58 proximal to each locking tab 62 so as to prevent the advancement member 48 from completely separating from the housing 12 when the advancement member is distally extended from the housing. This is accomplished by each stop surface 64 engaging with a respective one of the lock wedges 72, which prevents further distal movement of the advancement member. Thus, after full distal extension, the advancement member 48 is locked from either proximal movement to cause re-entry of the advancement member into the housing cavity 70 via engagement of the locking tabs 62 with the lock wedges 72, or further distal movement via engagement of the stop surfaces 64 with the lock wedges. In addition to these, other modes for preventing undesired proximal and distal movement of the advancement member after distal extension thereof can also be employed.

Note that in one embodiment the outer diameters of the needle 16 and the catheter tube 44 are lubricated with silicone or other suitable lubricant to enhance sliding of the catheter tube with respect to the needle and for aiding in the insertion of the catheter into the body of the patient.

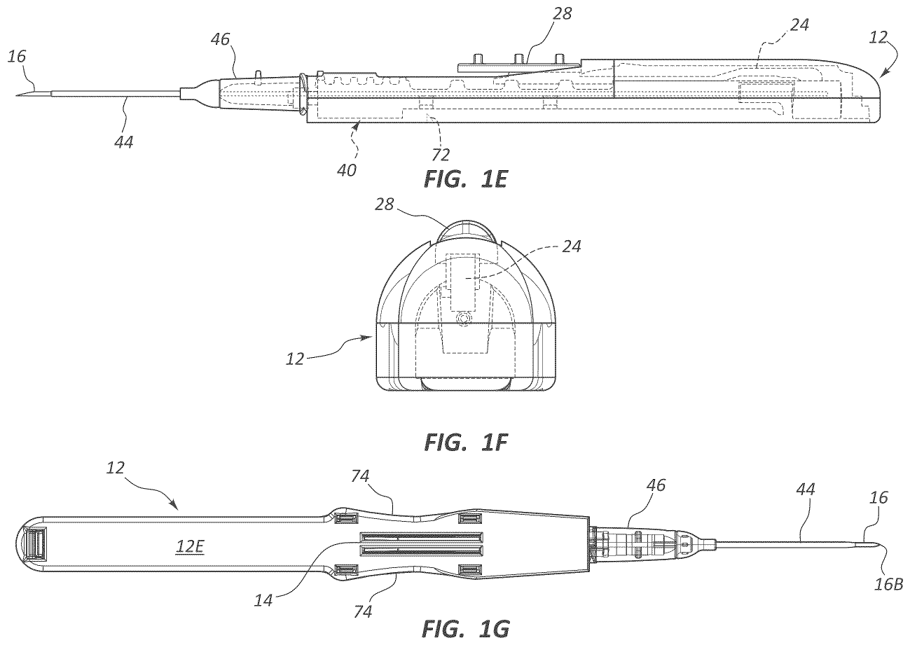

FIGS. 3A-3C depict various stages of use of the insertion device 10 in placing the catheter 42 in the vasculature of a patient. For clarity, the various stages are depicted without actual insertion into a patient being shown. With the insertion tool 10 in the configuration shown in FIG. 1E, a user grasping the insertion device 10 first guides the distal portion of the needle 16 through the skin at a suitable insertion site and accesses a subcutaneous vessel.

After needle access to the vessel is confirmed, the guidewire advancement assembly 20 is actuated, wherein the finger pad 28 (disposed in the slot 32 defined in the housing) is advanced by the finger of the user to distally advance the guidewire 22 (FIG. 3A), initially disposed within the hollow needle 16. Note that the guidewire 22 is distally advanced by the guidewire lever 24, which is operably attached to the slidable finger pad 28.

Distal advancement of the guidewire 22 continues until the finger pad 28 has been distally slid a predetermined distance, resulting in a predetermined length of the guidewire 22 extending past the distal end of the needle 16, as shown in FIG. 3A.

At this point, the finger pad 28 is slid distally an additional distance, which causes the push tab 30 (FIG. 2B) of the guidewire lever 24 to abut against the proximal end 56A of the ribs 56 of the advancement member 48 of the catheter advancement assembly 40. This in turn causes the advancement member 48 to distally advance out the open distal end of the housing 12D a predetermined distance. As it is removably attached to the distal portion 49 of the advancement member 48 via the hub engagement tabs 50, the catheter 42 is also distally advanced the predetermined distance. Distal advancement of the advancement member 48 ceases when the notch above the guidewire lever push tab 30 contacts the distal end of the slot 32 (FIG. 1I) defined in the housing 12 and stops further distal sliding of the finger pad 28.

Once the guidewire lever 24 has been fully distally extended via sliding of the finger pad 28, which in turn has extended the guidewire 22 past the distal end 16B of the needle 16 and into the vessel of the patient and has distally advanced the advancement member 48 and connected catheter 42 a predetermined distance away from the device housing 12, further manual distal advancement of the advancement member 48 is performed by a finger of the user via pushing against the advancement tab 54 and then the secondary tabs 60 of the advancement member, as seen in FIG. 3B. This causes the catheter tube 44 to slide over distal portions of the needle 16 and guidewire 22 and into the patient's vasculature via the insertion site. In light of this, it is appreciated that the finger pad 28 acts as a first member used to advance the guidewire 22, whereas the advancement tab 54 acts as a second member used to advance the catheter 42, in the present embodiment. It is appreciated that the finger pad 28 is distally slidable to a distal termination point that is proximate a proximal commencement point of the second member such that movement of a finger of the user from the finger pad 28 to the advancement tab 54 occurs without substantial repositioning of the finger, in the present embodiment.

The advancement member 48 and connected catheter 42 are manually distally advanced until the advancement member has been fully extended, as seen in FIG. 3B, i.e., the locking tabs 62 slide over and lock with the lock wedges 72 defined by the housing 12, thus locking the advancement member from further distal advancement. In this fully extended state of the advancement member 48, the distal portion 49 is disposed over the needle 16 and shielded by the ribs 56. Thus, the advancement member 48 serves as one example of a needle safety component, according to the present embodiment. The engagement of the above-mentioned locking tabs 62, as well as the stop surfaces 64, of the advancement member 48 with the lock wedges 72 prevents further distal or proximal movement of the advancement member, thus desirably ensuring continued shielding of the distal end 16B of the needle 16.

The above distal advancement of the advancement member 48 likewise distally advances the catheter tube 44 over the needle 16 and guidewire 22 and into the vessel of the patient until the catheter hub 46 abuts the insertion site of the needle through the skin. As the needle 16 is no longer disposed between them, the hub engagement tabs 50 compress radially inward, thus releasing the friction fit between the protuberances 52 and the interior surface of the catheter hub. This enables the catheter 42 to be separated from the advancement member 48, as shown in FIG. 3C. The catheter 42, now in place in the patient, can be prepared for use and dressed down, per standard procedures. Then insertion device 10 can be discarded.

In light of the above, it is appreciated that the guidewire advancement assembly 20 and the catheter advancement assembly 40 operate in conjunction with one another in the present embodiment and thus comprise together a master advancement assembly for placing the catheter 42. It is further appreciated that the master advancement assembly can include both or only one of a guidewire advancement assembly and a catheter advancement assembly in other embodiments.

It is noted that the device 10 is configured such that grasping of the device and advancement of the guidewire 22 and the catheter 42 can be performed by only one hand of the user. This is accomplished in the present embodiment by placing the grip surfaces 74 and the finger pad 28 in convenient locations for user grasping of the device 10, together with enabling the finger pad 28 and guidewire advancement assembly 20 to be used to advance the guidewire 22 and the catheter 42 a predetermined distance, followed by manual advancement of the advancement member 48 by a finger of the user. This can be performed by a single thumb, finger, or fingers of the user, in the present embodiment. Of course, other grasping and advancement configurations can be employed.

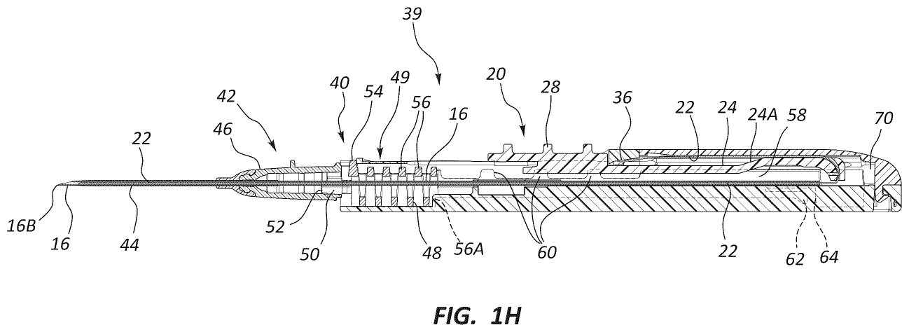

Reference is now made to FIG. 4, which shows a continuous blood flash indicator 80 that can be used with the device 10 according to one embodiment. The flash indicator 80 is employed to indicate the presence of blood in the lumen of the needle 16 during use of the device 10, thus assuring that proper access has been made by the needle into a vein or other desired blood-carrying vessel. As shown, the flash indicator 80 includes a translucent chamber 82 that is generally cylindrical in shape, sealed at either end, and disposed about a portion of the needle 16 such that the needle protrudes out from either sealed end. In the present embodiment the chamber 82 is disposed just distal to the needle hub 14 within the housing 12, though other locations along the needle are also possible.

Two notches—a first notch 83 and a second notch 84—are defined in the needle 16 so as to provide fluid communication between the lumen of the needle and the interior of the chamber 82. The notches 83 and 84 replace the notch 18 (FIG. 1I) in one embodiment, and are included in addition to the notch 18, in another embodiment. It is appreciated that, in one embodiment, blood passage through the notch 18 serves as an initial indicator that the distal end 16B of the needle has entered the vein, while the embodiment shown here serves as an additional indicator to verify that the needle distal end remains in the vein after initial access.

In the present embodiment, the second notch 84 is disposed just proximal to the distal termination point of the guidewire support tube 19, though other locations for the notches are possible. Also as shown, the guidewire 22 passes through the lumen of the needle 16 so as to extend through the flash indicator 80. The first notch 83 is disposed distal to the second notch 84 toward the distal end of the chamber 82, as shown in FIG. 4.

When vessel access is achieved by the distal end 16B of the needle 16, blood travels proximally up the lumen of the needle, between the inner surface of the needle and the outer surface of the guidewire 22, disposed in the needle lumen. Upon reaching the relatively more distal first notch 83 defined in the needle 16, a portion of the blood will pass through the first notch and enter the chamber 82. As the blood fills the chamber 82, a user can observe the translucent chamber through the translucent housing 12 of the insertion device 10 and view the blood therein, thus confirming that the vessel access has been achieved. In another embodiment, the housing 12 can be configured such that direct viewing of the chamber 82 is possible, e.g., with no intervening structure interposed between the chamber and the user.

The second notch 84 is employed to provide an exit point for air in the chamber 82 to equalize air pressure and enable the blood to continue entering the chamber via the first notch 83. It is noted that the spacing between the inner surface of the needle 16 and the outer surface of the guidewire support tube 19 is such that air but not blood can pass therebetween, thus enabling air pressure equalization in the chamber 82 without blood passage through the second notch 84. In this way, the flash indicator 80 is a continuous indicator, enabling a continuous flow of blood into the chamber 82 while the needle distal end 16B is disposed within the vessel.

Note that the chamber 82 of the flash indicator 80 of the present embodiment is disposed so as to be directly under the ribs 56 of the distal portion 49 of the as-yet un-advanced advancement member 48 during the establishment of needle access to the vessel. This enables the ribs 56 to act as an indicia, or an approximate blood flash meter, as the blood proceeds proximally within the chamber 82 of the flash indicator 80; a user observing the blood in the spaces between the ribs 56 proceeding proximally can view the proximal travel of the blood during the catheter placement procedure. In another embodiment, a spring disposed in the housing 12, such as for retraction of the needle 16, can also serve as indicia to meter the flow of blood in the flash indicator. These and other indicia for metering blood flow in the flash indicator are therefore contemplated.

Note that the catheter insertion device 10 can include more than one flash indicator. In one embodiment and as mentioned above, for instance, the blood flash indicator 80 can be included, along with another flash indicator, such as the notch 18, which enables blood present in the lumen 17 of the needle 16 to proceed proximally up the space between the outer surface of the needle and the inner surface of the catheter 42.

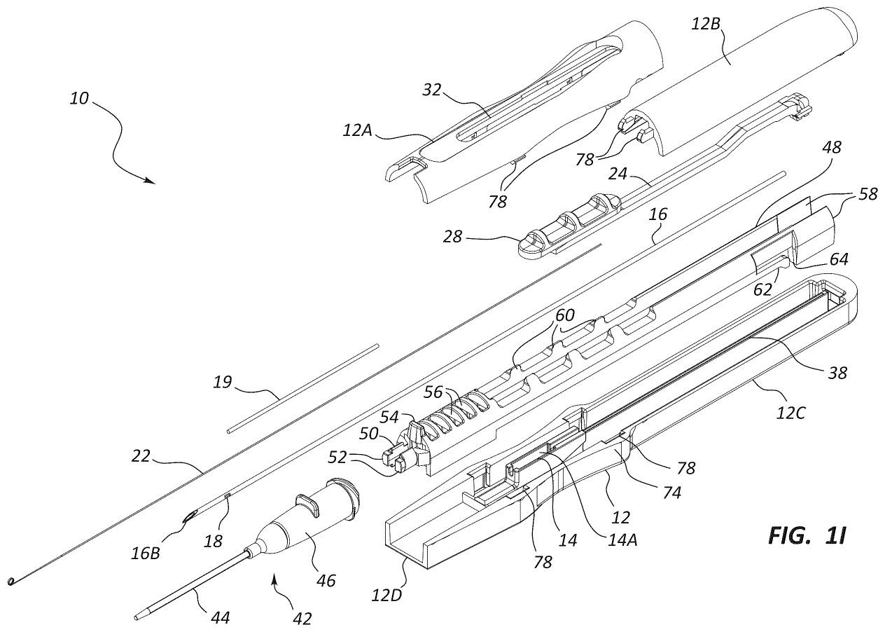

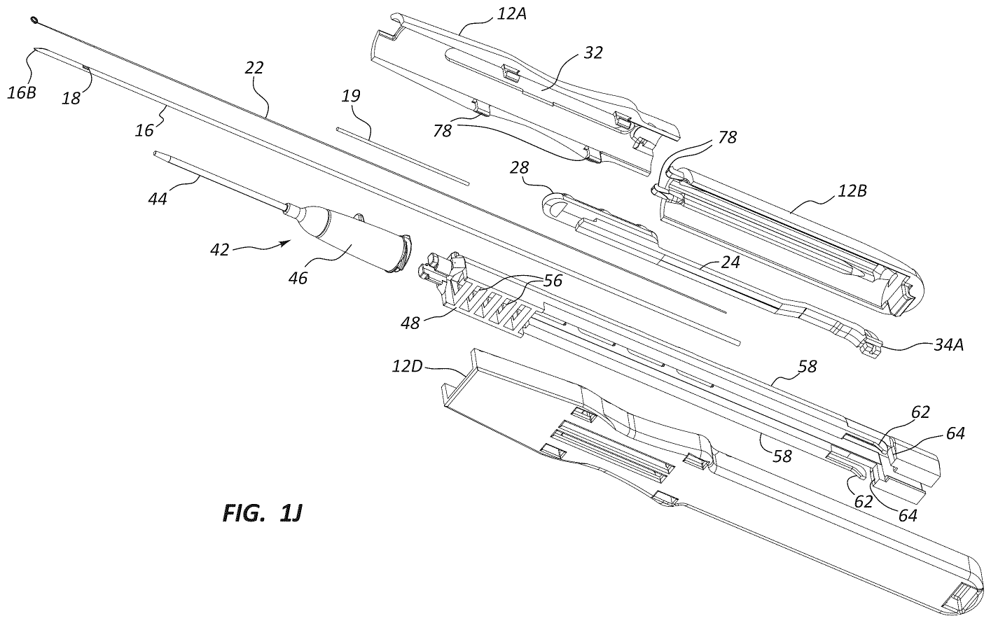

FIGS. 5 and 6 show another example of a continuous blood flash indicator 80 according to one embodiment, wherein an absorbent component 86 capable of absorbing blood or other desired body fluid is wrapped/disposed about a portion of the outer surface of the needle 16 so as to cover the notch 18 of the needle (FIGS. 1I, 1J). Cotton, gauze, fabrics, wood-based products, hydrophilic materials, mesh materials, polymeric materials, polyester, woven materials, and other suitable substances—both natural and synthetic materials—are examples of materials that can be employed for the absorbent component. In one embodiment, the absorbent material is woven to include a weave such that the blood fills the spaces between the woven material. This enables the amount of expansion of the absorbent material as well as the speed of blood travel through the absorbent material. Note that expansion of the absorbent material can be radial, longitudinal, a combination of both, etc.

The absorbent component preferably has a color different from red so as to indicate when blood has been absorbed. A translucent cover 88, including a thermoplastic or other suitable material, can be optionally placed over the absorbent component 86 in one embodiment to contain it and isolate blood absorbed thereby.

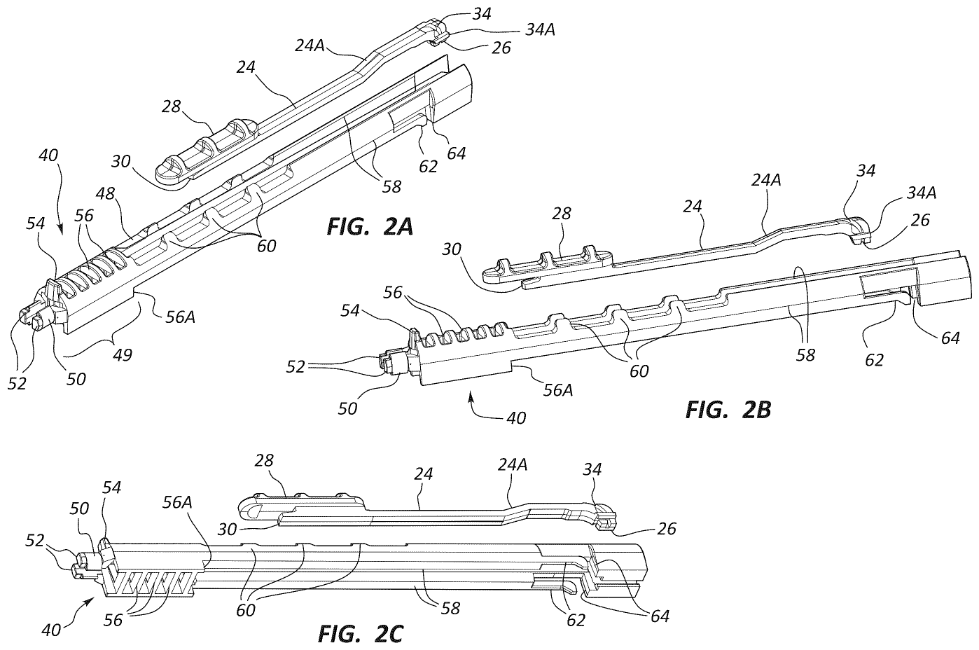

When vessel access is achieved by the distal end 16B of the needle 16, blood travels proximally up the lumen of the needle, between the inner surface of the needle and the outer surface of the guidewire 22, which is disposed in the needle lumen. Upon reaching the notch 18 defined in the needle 16, a portion of the blood will pass through the notch and be absorbed by the absorbent component 86, which changes colors due to the blood absorption. This indicates to the user that the needle distal tip 16B is properly located in the vessel. As the needle distal tip 16B remains in the vessel, blood will continue to be absorbed by the absorbent component 86, causing the absorbed blood to progress along the length of the absorbent component, thus providing a continuous blood flash indication. FIG. 7 shows that the diameter of the absorbent component 86 can vary in size; as such, modifications to what has been shown and described herein are therefore contemplated. In another embodiment it is appreciated that the absorbent material can expand in size as it absorbs blood or other fluid with which it is to be used.

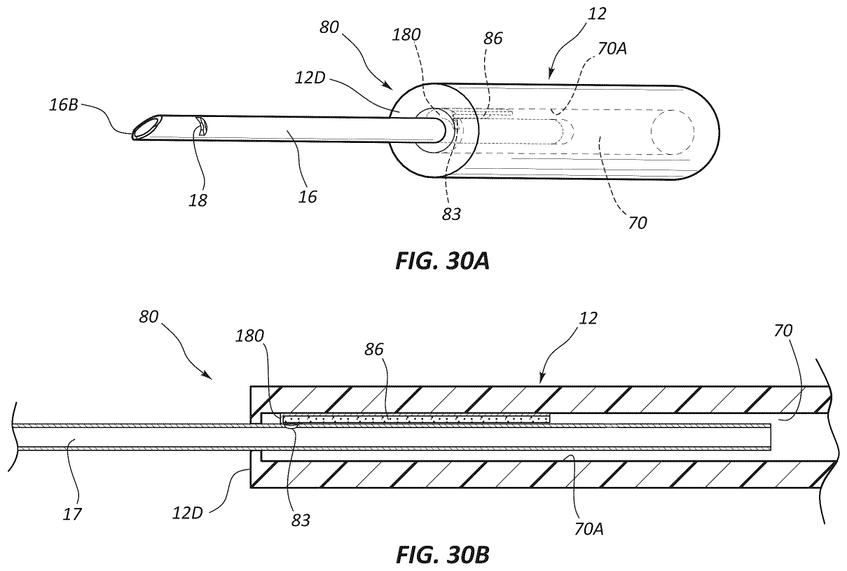

FIGS. 30A and 30B depict various details of a variation of the blood flash indicator 80 of FIGS. 5 and 6 wherein the absorbent component 86 includes a strip of absorbent material a first portion of which is disposed over a notch in the needle 16, such as the first needle notch 83. The first portion of the absorbent material strip is connected to a second portion of the absorbent material strip that longitudinally extends proximally from the first notch 83, though it may also extend in other directions and possess other shapes, sizes, etc. The second portion of the absorbent material strip is secured to an inner surface 70A of the housing cavity 70 and is connected to the first portion covering the first notch 83 via a tether 180, which also may comprise a portion of the absorbent material. So configured, the absorbent component 86 absorbs blood (or other fluid) that exits the lumen 17 of the needle 16 via the first notch 83, such as when the distal end 16B of the needle is disposed within a vein of the patient. As it continues to exit the first notch 83, the blood will be continually absorbed by the absorbent component 86 and travel from the first portion of the absorbent material, across the tether 180, and proximally along the strip of absorbent material, thus providing a continuous flash indicator to the observing user. The tether 180 is frangible in the one embodiment to enable the needle 16 to be retracted into the housing 12. Upon retracting the needle 16, the tether 180 will break, allowing the second portion of the absorbent material that is affixed to the housing cavity inner surface 70A to remain in place. In one embodiment, the flash indicator 80 described herein in connection with FIGS. 30A and 30B can be included in a flash chamber, such as the flash chamber 82 shown in FIG. 4, for instance. A variety of materials may be employed for the absorbent component, including those described in connection with FIGS. 5-7.

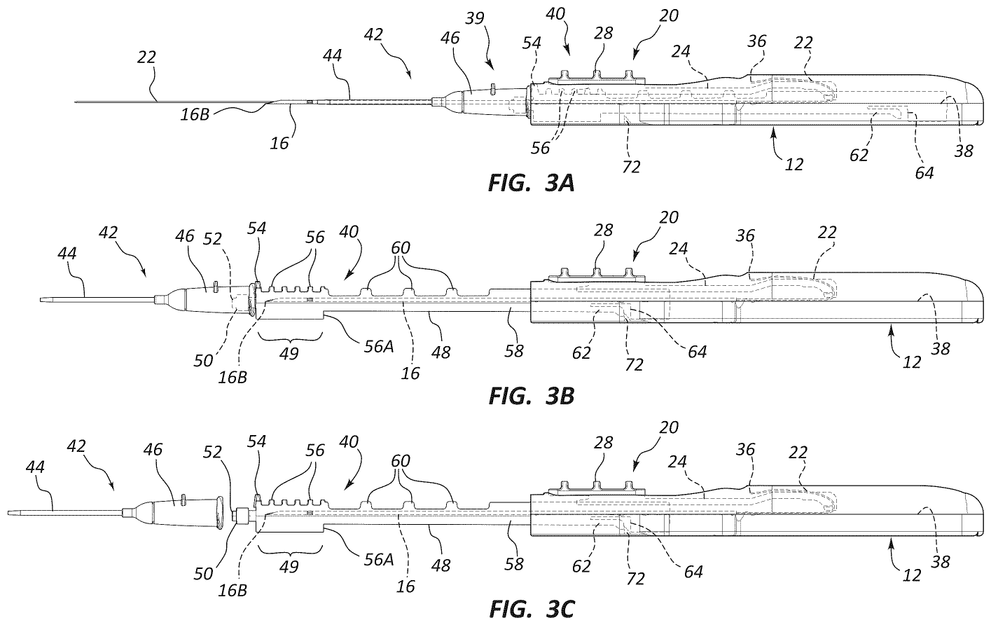

FIGS. 8A-8H depict details of the catheter insertion device 10 according to another embodiment, including the housing 12 from which distally extends the needle 16 secured in place by the needle hub 14 within the housing. The catheter 42 is removably disposed over the needle 16 such that the needle passes through the hub 46 and catheter tube 44, as shown. The guidewire 22 is initially disposed within the housing and the lumen of the needle 16 and is selectively advanceable.

The insertion device 10 includes an advancement assembly 120 for selectively advancing the guidewire 22 and the catheter 42. The advancement assembly 120 includes the finger pad 28 that is slidably connected with the housing 12. The finger pad 28 is part of a telescoping portion 90 and can be slid distally, as shown in FIGS. 8E and 8F, to distally advance the guidewire 22 out the distal end 16B of the needle 16. Once the guidewire 22 has been fully deployed distally, further distal sliding of the finger pad 28 causes a portion of the telescoping portion 90 to engage the catheter hub 46 (FIG. 8G) and move the catheter 42 distally until the telescoping portion is fully extended (FIG. 8H). At this point, the catheter 42 can be removed from the insertion device 10, at which point the telescoping portion 90 has extended sufficient to cover and shield the distal tip 16B of the needle 16, thus protecting the user from an unintended needle stick. Operation of the insertion device 10 of FIGS. 8A-8H as described herein enables the insertion device to be used in gaining access to a vessel in the body of a patient, deploying the guidewire and catheter into the vessel, and shielding the needle 16 after use, according to one embodiment. It is noted that the embodiment shown in FIGS. 8A-8H shows one example of an insertion device that enables full guidewire and catheter advancement using a single finger pad and telescoping component.

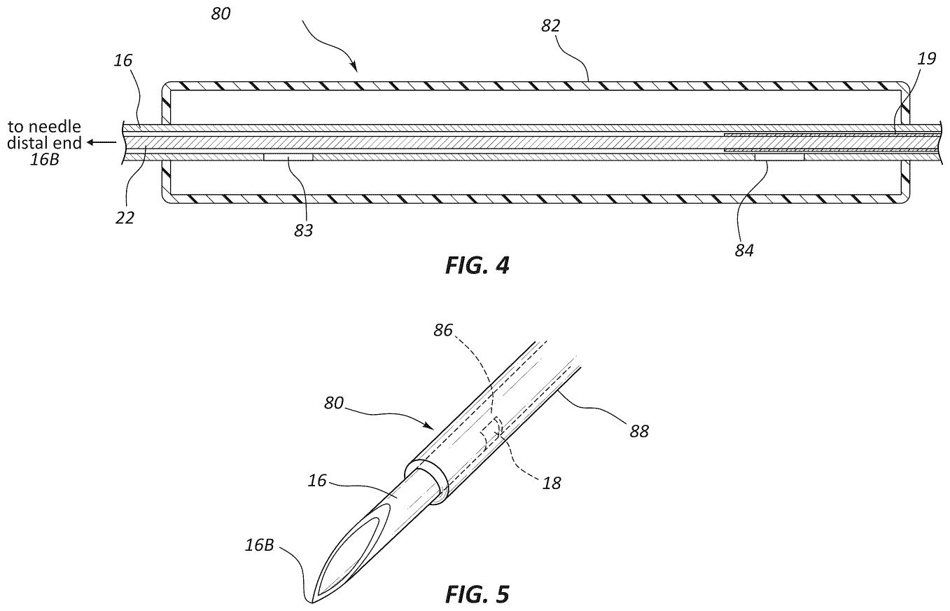

FIGS. 9A-9G depict details of a needle safety component 100 for use with a needle-bearing device, such as the insertion devices discussed herein. As shown, the needle safety component 100 is operably attached to the needle 16 and is disposed within a housing 101 (FIGS. 9E-9G). The needle safety component includes a locking element 102 that is implemented here as a flattened, elongate metal bar in which a wave-shaped spring element 104 is formed proximate one end and a hook portion 108, which acts as a cam follower, on an opposite end. A D-shaped hole 106 is defined through a central portion of the locking element 102 and the needle 16 initially passes through the hole and through the catheter 42, as shown in FIGS. 9A and 9B. Other hole shapes are also possible.

A cam 110 is rotatably within the housing 101, as shown in FIG. 9E, and includes a hub engagement portion 112, a biasing portion 114, and a locking portion 116. Before actuation, the needle safety component 100 is configured as shown in FIG. 9E, with the needle safety component disposed over the needle 16 such that the needle passes through the hole 106 and the cam 110 rotated such that the hub engagement portion 112 thereof engages a threaded portion of the hub 46 of the catheter 42 so as to maintain engagement between the catheter and the needle safety component. The biasing portion 114 of the cam 110 also engages a portion of the catheter hub 46 to help maintain engagement between the hub and the cam. Note that the spring element 104 is compressed against an interior surface of the housing 101 and the hook portion 108 of the locking element 102 is disposed in the locking portion 116 of the cam 110.

Once the catheter 42 has been positioned in the patient, the needle 16 is withdrawn from the catheter and retracted from the hole 106 in the locking element 102, while the catheter hub 46 separates from engagement with the cam 110, causing the cam to rotate counterclockwise. This rotation of the cam 110 causes the hook portion 108 of the locking element 102 to exit the locking portion 116 of the cam 110 and slide up against the side of the cam, as seen in FIG. 9F. This enables the spring element 104 to un-compress from its compressed state (shown in FIG. 9E), thus moving the hole 106 out of alignment with the needle 16 (FIG. 9G) and desirably preventing the ability of the needle to re-emerge from the housing 101. The distal tip 16B of the needle 16 is thus shielded from the user.

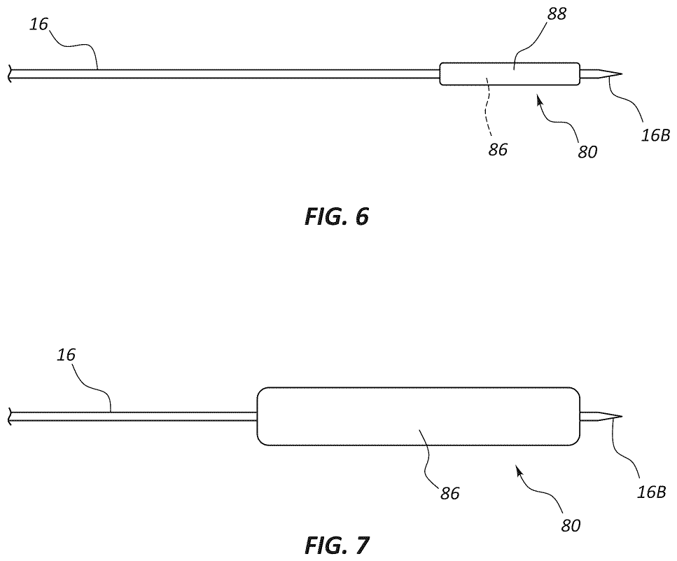

FIGS. 10-12 depict various examples of configurations of the guidewire 22 to enable blood to more easily pass through a lumen 17 of the needle 16, according to example embodiments. In the cross-sectional view of FIG. 10, for example, the guidewire 22 is shown disposed in a lumen 17 of the needle 16. The guidewire 22 defines a flattened bar, or generally rounded rectangular, cross-sectional shape, which frees up space within the needle lumen 17 for the passage of blood therethrough, such as when the distal tip 16B of the needle 16 enters a vein or other blood-carrying vessel of the patient. This, in turn, helps blood to flow into a blood flash indicator, such as those shown and described herein.

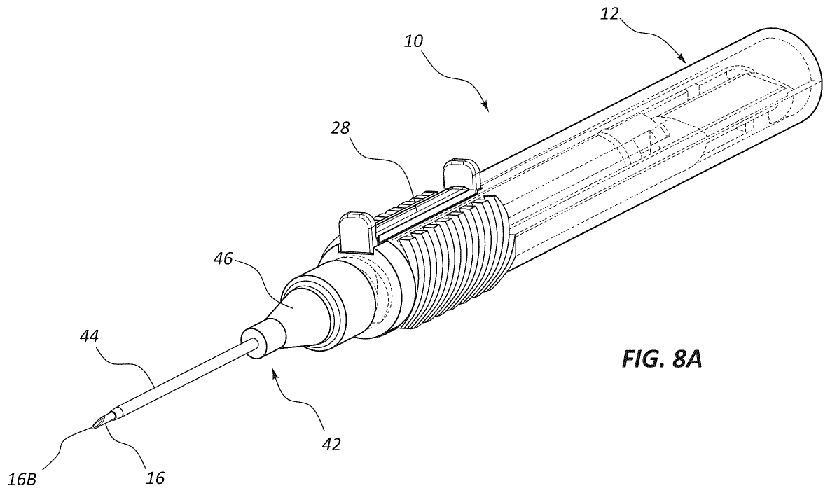

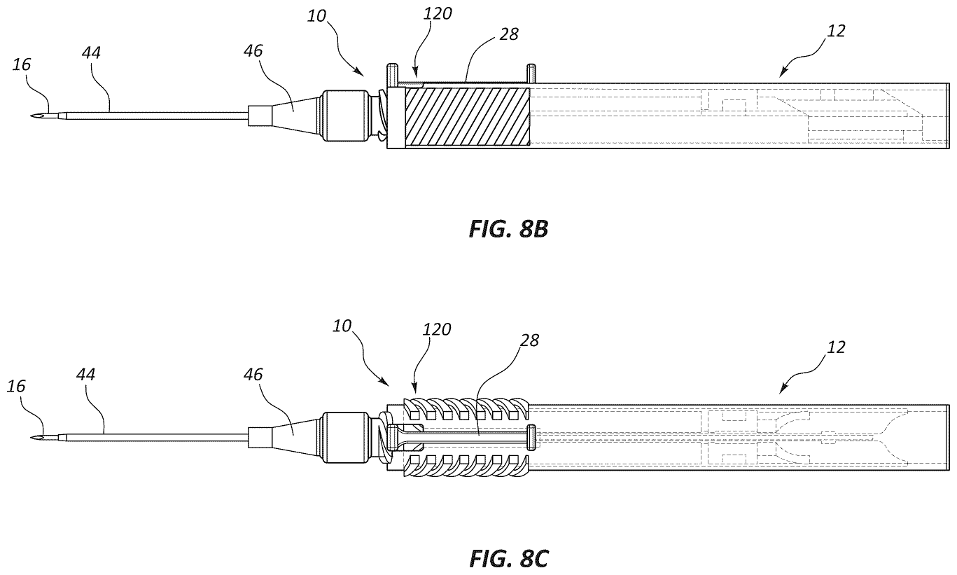

FIG. 11 shows a cross-sectional configuration of the guidewire 22 according to another embodiment, wherein the cross-sectional view of the guidewire depicts two longitudinal notches 117 defined in the guidewire profile. FIG. 12 shows three notches 117 defined in the cross-sectional profile of the guidewire 22. These and other guidewire configurations are therefore contemplated.

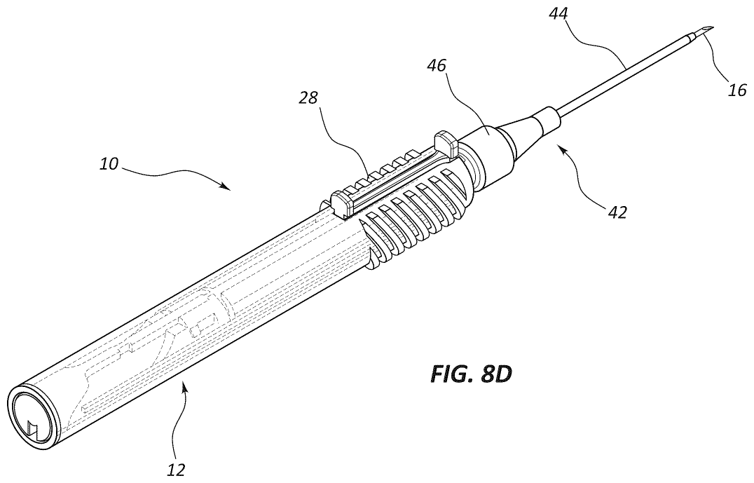

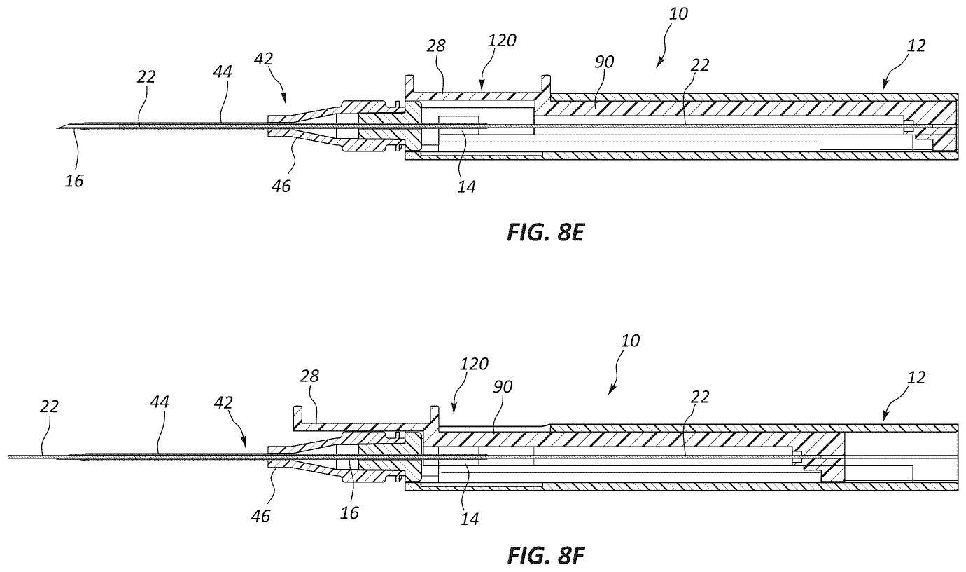

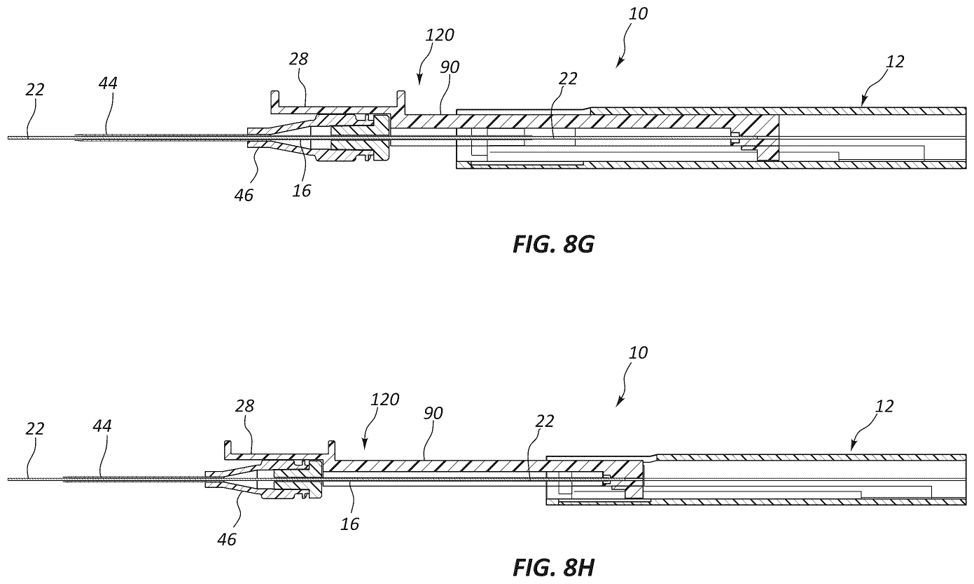

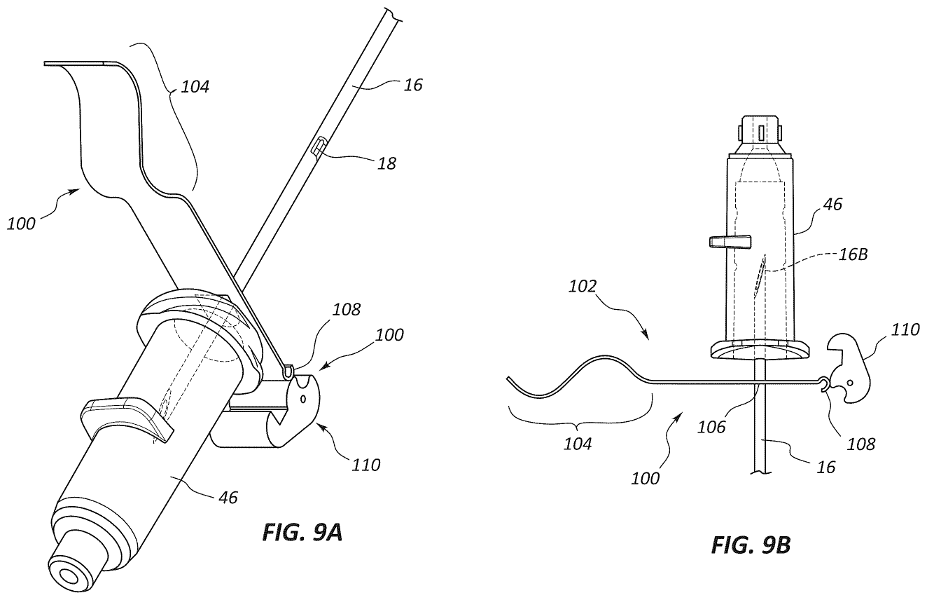

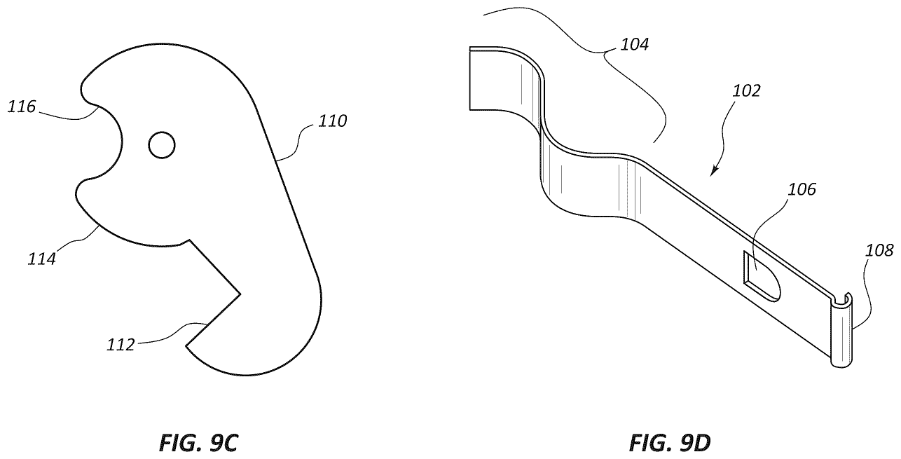

FIGS. 13-17 depict various examples of configurations of a distal end 22B of the guidewire 22 that are designed to provide an atraumatic tip to prevent damage to the vessel during catheter insertion procedures. For example, FIG. 13 shows the guidewire 22 extending out the distal tip 16B of the needle 16. The distal portion of the guidewire 22 includes a curled configuration proximate the distal guidewire end 22B. FIG. 14 shows a J-tipped guidewire configuration wherein the guidewire distal end 22B is doubled back on itself to form a J-tip. In FIG. 15, a thermoplastic or metallic ball 122 is secured to the distal end 22B of the guidewire 22. In FIG. 16, a three-dimensional whisk-like tip 126 is provided at the distal end 22B of the guidewire 22. FIG. 17 shows another configuration, wherein the guidewire distal end 22B includes a ball attached thereto, and the distal portion of the guidewire 22 is in a curled configuration. These and other modifications to the guidewire 22 are therefore contemplated. Note that in one embodiment, the guidewire 22 is composed of nitinol or other suitable material, as may be contemplated.

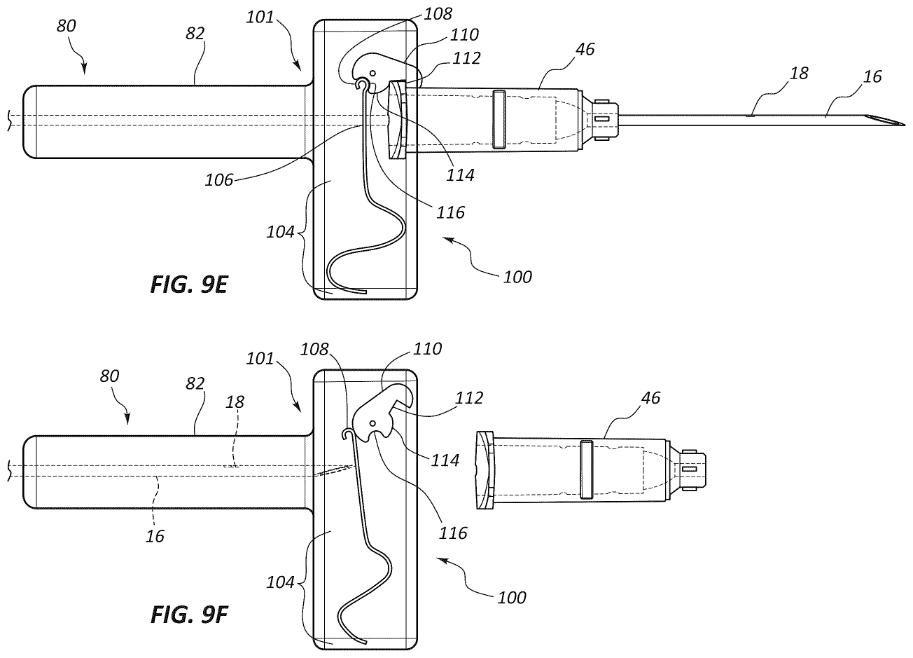

FIG. 18 depicts the advancement member of the catheter insertion device 10 according to another embodiment, wherein the advancement member includes outwardly-extending wings 130 on a proximal end thereof, which are configured to prevent re-entry of the advancement member into the housing of the insertion device after full extension of the advancement member has been performed. This is but one example of modes by which unintended advancement member re-entry can be prevented.

FIG. 18 further depicts a continuous blood flash indicator 80 according to one embodiment, wherein an elongate channel 134 is defined on a surface portion of the advancement member 48 so as to be in fluid communication with the lumen of the needle 16 of the insertion device 10. The channel 134 is shaped as to define a pathway 136, such as a tortuous pathway for instance, along which blood present in the lumen of the needle 16 can travel after exiting the needle. The pathway 136 shown in FIG. 18 defines a back-and-forth pattern along a top portion of a cylindrical segment of the advancement member 48, though a variety of different pathway designs can be employed. A user can observe the blood within the pathway 136 defined by the channel 134 to confirm that the distal tip of the needle 16 is disposed in the vein or other desired blood-carrying vessel of the patient. As the pathway 136 is relatively lengthy, the progress of the blood as it proceeds in the channel enables the flash indicator 80 to function as a continuous flash indicator. It is appreciated that the channel and pathway can be formed with one of a variety of processes, including molding, machining, etc.

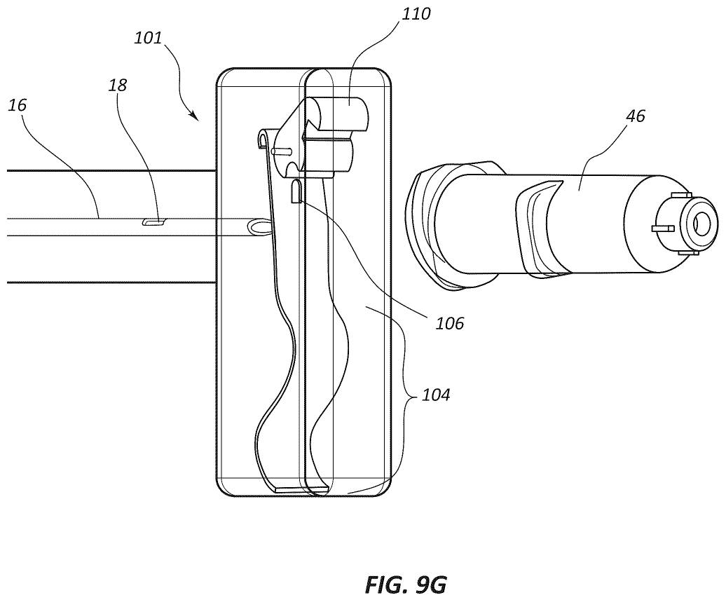

In light of the above, FIG. 19 depicts the continuous blood flash indicator 80 according to another embodiment, wherein the channel 134 defines a wavy, back-and-forth pathway 136 on a surface of the advancement member. As before a distal end of the channel 134 is in fluid communication with the lumen of the needle of the insertion device (or other medical device) so that blood may exit the needle lumen and enter the pathway 136 defined by the channel. Though shown here as being defined on the advancement member 48 of the catheter insertion device, the channel 134 can be included on other structures, including the hub or other portion of the catheter, the housing of the catheter insertion device/medical device/component, a valve assembly, etc. Further, though shown here as defined on a surface of the advancement member, the channel in other embodiments can include at least a portion of a tunnel or pathway defined below the surface of the advancement member/medical device/component. The pathway can be covered by a translucent or other cover, in another embodiment.

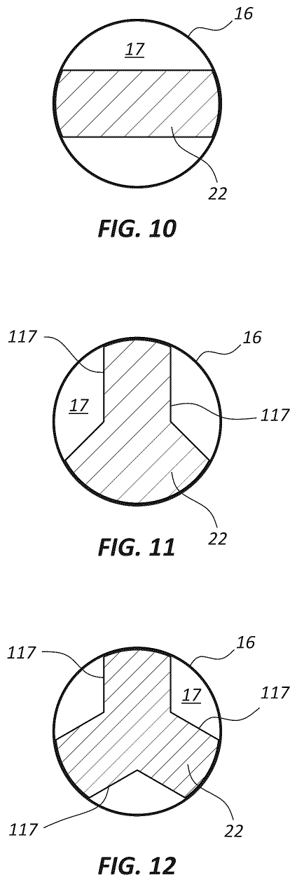

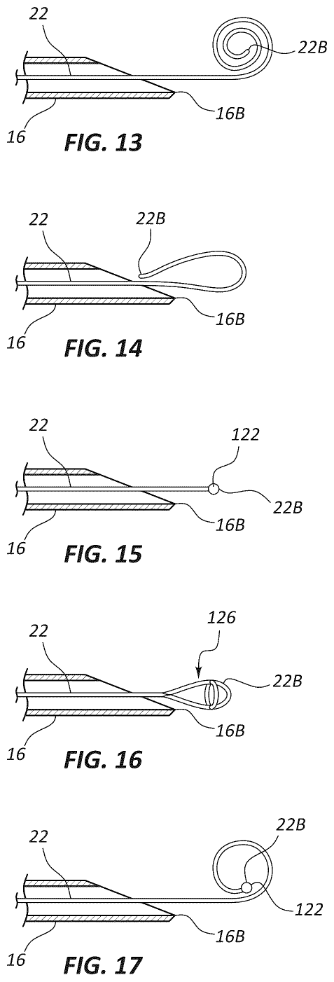

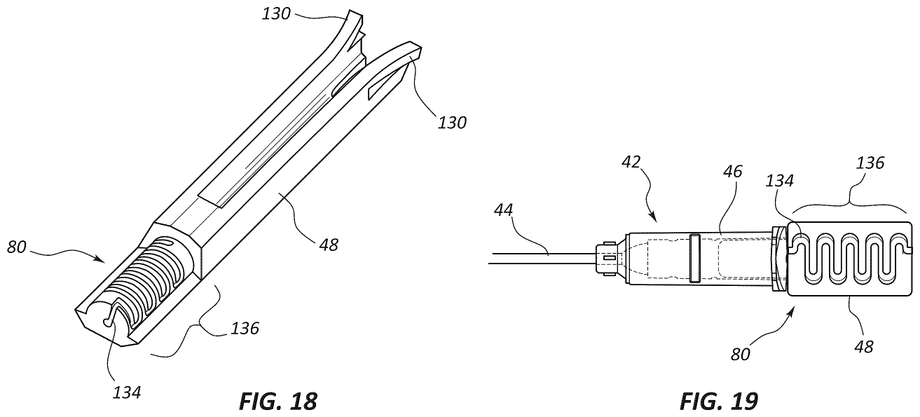

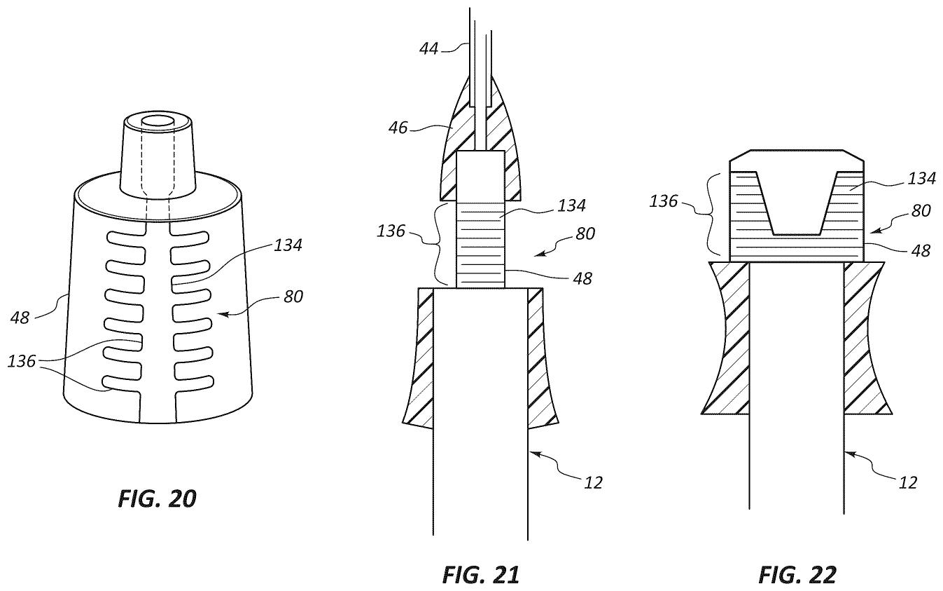

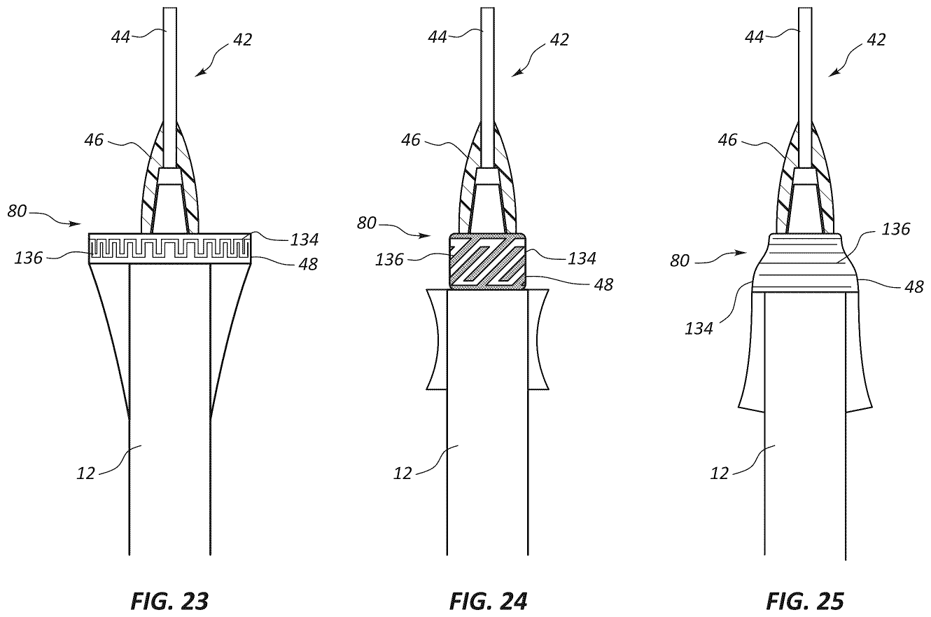

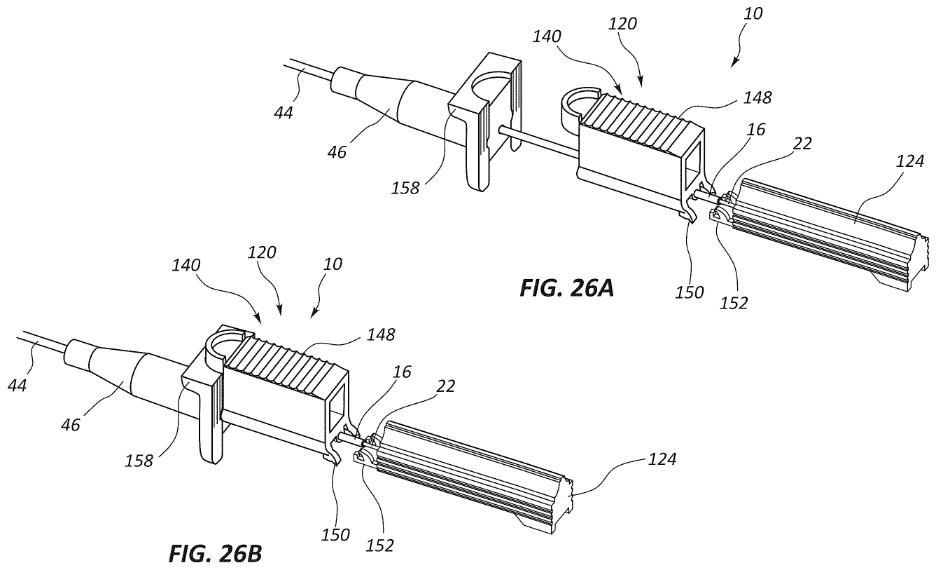

FIG. 20 depicts the continuous blood flash indicator 80 according to another embodiment, wherein the channel 134 defines a trunk-and-branch pathway 136. In FIGS. 21 and 22, the advancement member 48 includes the channel 134 defining a back-and-forth pathway 136 on a relatively thin distal portion (FIG. 21) and a relatively thick portion (FIG. 22) of the advancement member. FIG. 23 depicts the channel 134 as defining a tooth-like pathway 136 about a circumference of a distal portion of the advancement member 48, while FIG. 24 shows the channel defining an angled zig-zag pattern thereon. And in FIG. 25, the channel 134 defines a converging back-and-forth pathway. Thus, these and other pathway designs, including circular, helix, etc., are contemplated.

It is appreciated that various other configurations are contemplated for the continuous blood flash indicator 80 and its shaped pathway. For instance, the flash indicator can be included on/with the catheter hub 46 such that blood travels through the lumen 17 of the needle 16 to the catheter hub and into the flash indicator. In yet another embodiment, the flash indicator 80 is included as a removable piece temporarily attached to the catheter hub 46 or other component of the insertion device 10. It is further appreciated that the flash indicators described herein can also be used to indicate the presence of other fluids in the needle, including other bodily fluids, for instance. These and other modifications are contemplated.

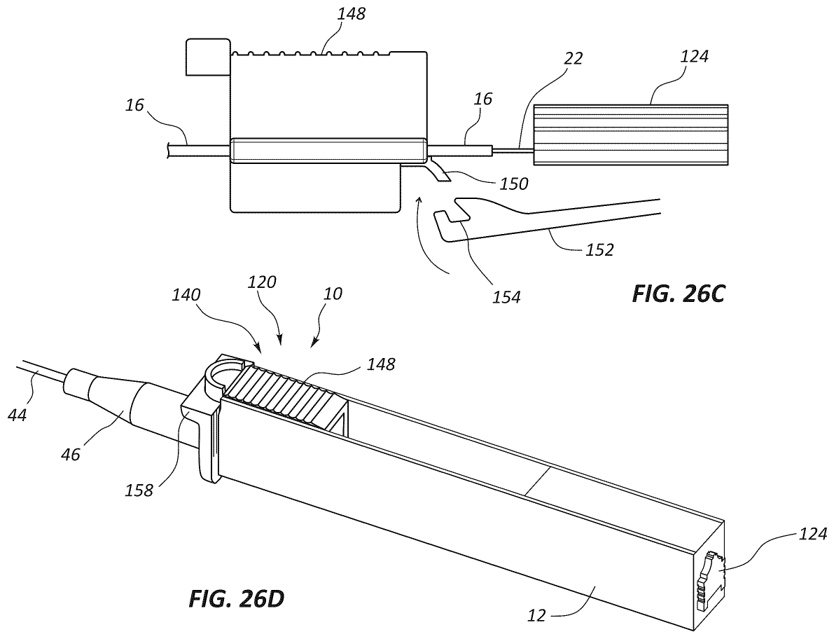

FIGS. 26A-26D depict details of the catheter insertion device 10 according to another embodiment, wherein the housing 12 has been removed from the views in FIGS. 26A-26C for clarity. The insertion device 10 here includes the needle 16 distally extending from the housing 12, with the catheter 42 removably disposed over the needle such that the needle passes through the hub 46 and catheter tube 44. The guidewire 22 is initially disposed within the housing and the lumen of the needle 16, and is selectively advanceable.

The insertion device 10 includes the advancement assembly 120 for selectively advancing the guidewire 22 and the catheter 42. The advancement assembly 120 includes an advancement slide 140, which in turn includes a finger pad 148 that is slidably connected with the housing 12. A guidewire carriage 124 is also included, from which distally extends the guidewire 22 to enter the needle 16. An end piece 158 is also included at the distal end of the housing 12 and serves to push the catheter 42 distally, as will be discussed.

The initial position of the catheter insertion device 10 is shown in FIG. 26A (excluding the omitted housing 12, for clarity), wherein the advancement slide 140 and the guidewire carriage 124 are coupled together, as shown in FIG. 26C. In detail, an arm 152 distally extending from the guidewire carriage 124 couples with an angled tab 150 of the advancement slide 140 via a notch 154 defined by the arm. Note that the arm 154 is downwardly biased, which would cause the arm to disengage the notch 154 from the angled tab 150; however, the housing 12 is shaped in the position shown in 26A to constrain the arm to maintain engagement with the angled tab.

The above configuration enables joint distal movement of the advancement slide 120 and the guidewire carriage 124 when the uses manually engages the finger pad 148 and slides the advancement slide distally to the position shown in FIG. 26B. This movement causes the guidewire 22 to fully extend out the distal end 16B of the needle 16, as with other embodiments. At this point, the notch 154 of the downwardly-biased arm 152 disengages with the angled tab 150 due to the arm 152 no longer being constrained by the housing 12 (or other suitable structure) to maintain the engagement with the angled tab. Thus, further distal movement of the guidewire 22 by the guidewire carriage 124 is prevented. A locking mechanism can be included to lock the guidewire carriage 124 in place, in one embodiment. Reversing the process by proximally sliding the advancement slide 120 causes the guidewire carriage 124 to re-couple with the advancement slide via re-coupling of the notch 154 of the arm 152 with the angled tab 150.

Next, further distal sliding of the advancement slide 120 via the finger pad 148 causes the advancement slide to engage the end piece 158, which in turn distally advances the catheter 42 off the needle 16 and into the patient, as desired. Once fully distally extended, the advancement slide 140 shields the distal tip 16B of the needle 16, thus protecting the user. This embodiment thus shows an example of an insertion device that enables full guidewire and catheter advancement using a single advancement assembly.

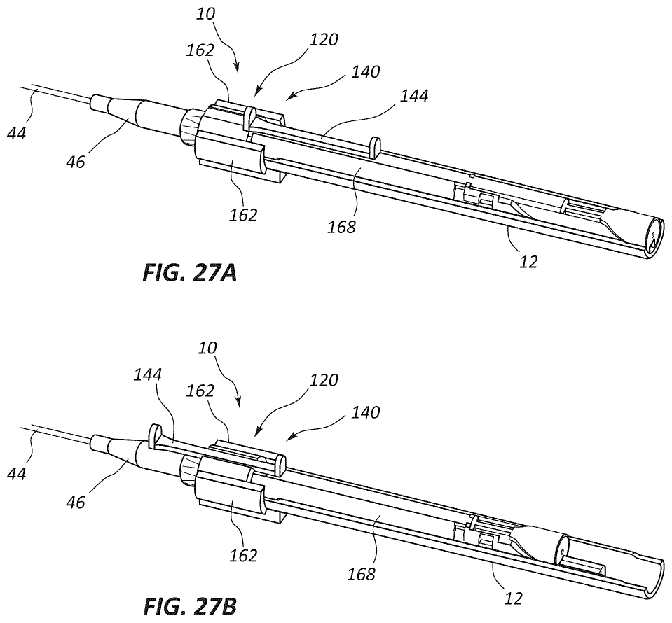

FIGS. 27A-27E depict details of the catheter insertion device 10 according to another embodiment, wherein a top portion of the housing 12 has been removed for clarity. The insertion device 10 here includes the needle 16 distally extending from the housing 12, with the catheter 42 removably disposed over the needle such that the needle passes through the hub 46 and catheter tube 44. The guidewire 22 is initially disposed within the housing and the lumen of the needle 16, and is selectively advanceable.

The insertion device 10 includes the advancement assembly 120 for selectively advancing the guidewire 22 and the catheter 42. The advancement assembly 120 includes a guidewire lever, which in turn includes the finger pad 148 that is slidable along a portion of the length of the insertion device housing 12. Two grips 162 are disposed proximate the distal end of the insertion device 10 to assist with user grasping of the insertion device.

The insertion device 10 further includes a catheter slide 168 that is distally slidable within the housing 12 to distally advance the catheter 42 during use of the insertion device for catheter insertion procedures. The catheter slide 168 includes a pair of wings 166 that interact with the guidewire lever 144 to enable catheter advancement after the guidewire 22 has been fully advanced.

The initial position of the catheter insertion device 10 is shown in FIG. 27A (excluding the omitted top housing portion of the housing 12, for clarity), wherein the guidewire lever 144 and its finger pad 148 have not yet been distally advanced. Distal sliding of the finger pad 148 manually by the user causes the guidewire lever 144 to distally advance the guidewire 22 out the distal end 16B of the needle 16, as with other embodiments. Once the guidewire 22 has been fully extended, a thinned portion 164 of the guidewire lever 144 is now positioned adjacent the wings 166 of the catheter slide 168, as seen in FIG. 27B. So positioned, the thinned portion 164 of the guidewire lever 144 enables the wings 166—which until this point were forced radially outward by the guidewire lever (FIG. 27C)—to contract radially inward (FIG. 27D) so as to enable the catheter slide 168 to be distally advanced by further distal sliding movement by the finger pad 148 of the guidewire lever, as seen in FIG. 27E. This distal advancement of the catheter slide 168 causes the catheter 42 to be distally advanced off the needle 16 and into the patient, as desired. This embodiment thus shows another example of an insertion device that enables full guidewire and catheter advancement using a single advancement assembly.

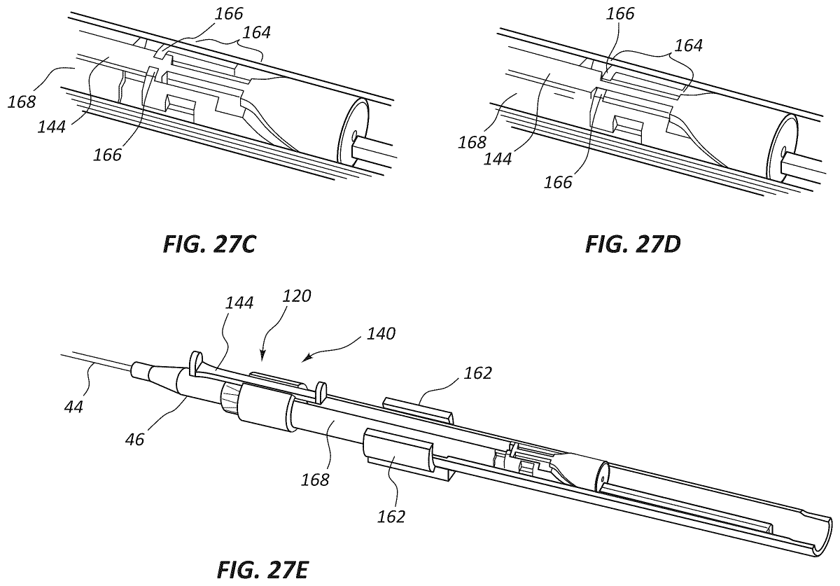

FIG. 28 depicts the continuous blood flash indicator 80 of the catheter insertion device 10 shown in FIGS. 27A-27E, including the channel 134 defining the pathway 136 implemented as a spiral disposed within the housing 12 about a distal portion of the advancement assembly 120.

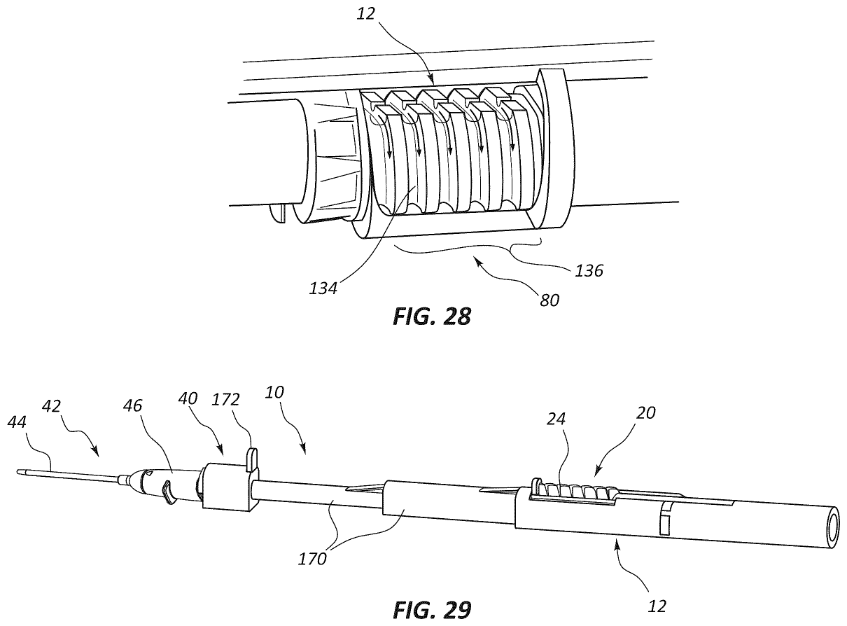

FIG. 29 depicts details of the catheter insertion device 10 according to another embodiment, including separately deployable assemblies, i.e., the guidewire advancement assembly 20 including the guidewire lever 24, and the catheter advancement assembly 40 including a tab 172 and a plurality of telescoping segments 170. During use of the insertion device 10, the guidewire 22 is distally extended manually via the guidewire lever 24, after which the catheter 42 is distally extended manually via the catheter advancement assembly 40 and the included tab 172. The telescoping segments 170 maintain attachment of the catheter advancement assembly 40 with the housing 12 and also help shield the needle from unintended user contact. Note that, though two are shown here, one, three or more telescoping segments 170 can be employed. This embodiment thus shows an example of an insertion device that enables full guidewire and catheter advancement using a separate advancement assembly for the guidewire and the catheter.

It is noted generally that, in one embodiment, the advancement assembly/assemblies can be configured to prevent distal advancement of the catheter until full distal advancement of the guidewire has been achieved. In other embodiments, a single advancement assembly is employed to advance both the guidewire and the catheter. For instance, the finger pad of an advancement assembly can be moved a first distance to distally advance the guidewire, after which further guidewire advancement is automatically or otherwise disengaged/prevented and the catheter distal advancement is commenced as the finger pad is moved a second distance, as in FIGS. 26A-26D. In another embodiment, a single finger pad is moved a first distance to distally advance the guidewire, then moved a second distance to continue moving the guidewire distally while also now moving the catheter distally as well, such as in FIGS. 27A-27E. These and other possible configurations are contemplated.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A needle assembly, comprising:
a needle defining a needle lumen, the needle having a proximal section disposed in a housing;
a notch defined in a side wall of the needle to enable blood to pass therethrough from the needle lumen when an open distal tip of the needle is disposed in a blood-carrying vessel of a patient; and
a flash indicator configured to indicate the presence of fluid within the needle lumen, the flash indicator including an absorbent material disposed about the needle and configured to absorb fluid that passes from the needle lumen via the notch such that the absorbed fluid is observable by a user of the needle assembly, the absorbent material including:
a first portion disposed proximate the notch;
a second portion attached to an inner surface of the housing; and
a tether portion interconnecting the first and second portions of the absorbent material.

2. The needle assembly according to claim 1, wherein a distal portion of the needle is retractable into the housing, the tether portion configured to break upon retraction of the needle such that the second portion of the absorbent material remains on the inner surface of the housing.

3. The needle assembly according to claim 1, wherein a portion of the housing proximate the second portion of the absorbent material is translucent to enable the user to view the fluid absorbed by the second portion of the absorbent material.

4. The needle assembly according to claim 1, further comprising a needle safety component, the needle safety component comprising:
a locking element comprising:
a hole through which the needle initially passes;
a spring element; and
a cam follower component; and
a cam component configured to operably engage with the cam follower component, the cam component movable between a first position wherein the cam compresses the spring element such that the hole of the locking element is aligned with a longitudinal axis of the needle, and a second position wherein the cam no longer compresses the spring element such that hole of the locking element mis-aligns with the longitudinal axis of the needle once the needle is withdrawn from the hole so as to prevent re-insertion of the needle through the hole.

5. The needle assembly according to claim 4, wherein the locking element of the needle safety component includes a flattened elongate bar, and wherein the cam follower component includes a hook portion disposed at a first end of the elongate bar.

6. The needle assembly according to claim 5, wherein the spring element of the needle safety component includes a wavelike-shaped portion of the elongate bar disposed proximate a second end of the elongate bar.

7. The needle assembly according to claim 4, wherein the cam component of the needle safety component in the first position engages a portion of a catheter assembly disposed on a portion of the needle and wherein the cam component is released from engagement with the portion of the catheter assembly when in the second position.

8. The needle assembly according to claim 7, wherein the portion of the catheter assembly engaged by the cam component in the first position includes a hub of the catheter assembly.

9. The needle assembly according to claim 1, wherein the absorbent material is configured such that blood continuously moves along the absorbent material while the open distal tip of the needle is disposed in the blood-carrying vessel of the patient.

10. The needle assembly according to claim 1, wherein the absorbent material is generally cylindrically shaped and includes at least one of a natural material and a synthetic material.

11. The needle assembly according to claim 1, wherein the absorbent material extends proximally from the notch along an outer surface of the needle.

12. The needle assembly according to claim 1, wherein the absorbent material includes cotton and is covered by a cover.

13. The needle assembly according to claim 12, wherein the cover includes a polymeric material.

14. The needle assembly according to claim 1, wherein the absorbent material is configured to expand in at least one of radial direction and a longitudinal direction upon absorption of a fluid.

15. The needle assembly according to claim 1, wherein indicia are placed in proximity to the absorbent material and are configured to meter a quantity of blood absorbed by the absorbent material.
